SEQUENCE LISTING (7 sequences)

SEQ ID NO: 1 - protein, 473 aa, synthetic construct
  REGION 1..473: note = Amino acid sequence of Serine palmitoyl-transferase
MATATEQWVLVEMVQALYEAPAYHLILEGILILWIIRLLFSKTYKLQERSDLTVKEKEELIEEWQPEPLVPPVPKDHPALNYNIVSGPPSHKTVVNGKECINFASFNFLGLLDNPRVKAAALASLKKYGVGTCGPRGFYGTFDVHLDLEDRLAKFMKTEEAIIYSYGFATIASAIPAYSKRGDIVFVDRAACFAIQKGLQASRSDIKLFKHNDMADLERLLKEQEIEDQKNPRKARVTRRFIVVEGLYMNTGTICPLPELVKLKYKYKARIFLEESLSFGVLGEHGRGVTEHYGINIDDIDLISANMENALASIGGFCCGRSFVIDHQRLSGQGYCFSASLPPLLAAAAIEALNIMEENPGIFAVLKEKCGQIHKALQGISGLKVVGESLSPAFHLQLEESTGSREQDVRLLQEIVDQCMNRSIALTQARYLEKEEKCLPPPSIRVVVTVEQTEEELERAASTIKEVAQAVLL

SEQ ID NO: 2 - DNA, 1713 bp, synthetic construct
  misc_feature 1..1713: note = polynucleotide sequence of LCB1
  source 1..1713: mol_type = other DNA
atgaacgtcactgctacaactataacaacttcaacaacaacaattgcattacaagatatttggaatacaacttctgatgttgtttctcgttatttattcattatattaaattatattgaattaatacctggtggttcaattttagttcgttatataaaatcttctcataaaaatgatccaattagaactttatttgaaattgctttatttatttttgcaattagatattttactacagcaaaatatgaaagatctaaaaaagatcatattaaattgaaaaattctgaaattgatgaattaattgatgattggatgccggaacctttagttttggatattagtccaaaggaacaatggcaattaaattcaattccaattgttaaaggtccaatagatactaaagtgaacctagttggtgaagaaggtgactttttaaattttgcttcttcaaattttttaaattttggtattaatccaattgttaaaaatgaatgtaaaaaaattattcatagtaatggtgttggtgcttgtggtccaccaaatttttatggtaatcaagatattcatattaaattagaaaatgatttagcaaaatttttcgaagttggtggtgctgtattatatggtcaagatttttgtactgcaggttcagttttaccaagttttttaaaaagaggtgattttgttattgctgatgcttcatcaaatgttgcaattcaaaaagctttacaattatcaagatgtgaaatttattggtttaatcataatgatttggatcatttagaagaaattttaattgatttacaaaaaaatatttttaaatttgaaaaaccaatttcaagaaaatttattgttactgaaggtatttttgcaaataaaggtgattcaccatatttaccaagattaattgaattaaagaaaaaatttaaatttagattatttttggatgaatctttatctttaggtgttttaggtaaatctggtaaaggtttagctgaacattataatattaaaagatcagaaattgatgtaactataagttcaatggctaattcattctcttcttcaggtgctttttgtattggtgataaagttatgacttatcatcaaagaattggttcaatggcttattgttttagtgcttcattacctgcttatgttgcaagagctacatcagttgcattaagattattaactgattctcaagattcccagggtgaatcatcaattgtaaaaaaattacaatcaaataattatcaattatttaatttatttaataaagatagaaaattaagtaaatatttaaaaattatatcaaatgaaatttcaccaattttacattttgaaattaattcagatttaagaaaacttttaaatttcccaattagttatacaggtaaaggatcagaaattgaatataaaaataaaaaaggaatttctgataaatttgttgaatcatttaattatgaaaatttaatttttcaaaaaattataaatttatccaagaaacaaggtattttaataacaagatcaatttttacaattgaacaagaagctctgcctctgattccaaatttaaaaattcattcaaatgttgattttactaaggatgaaattgaaaaagtttataaaattgtttccaaagtaattttagatgtttttgaaaatttaactgttgaatcattatcattattaactgaagaagttatttaa

SEQ ID NO: 3 - DNA, 1689 bp, synthetic construct
  misc_feature 1..1689: note = polynucleotide sequence of LCB2
  source 1..1689: mol_type = other DNA
atgtcattggtaatacctcaaatagatctatcaggtctttccatcgaagacaagaaacaaaatgaattcggtgctctaacttcaaatgaatatcgttacaaaacaatttcaagacaggggaaaccattacctgatccaattgaagatgaaccaccatatcatgtccttttcatcacttatttaaactatttaatcttgattatcgttggtcatattaaagatttcacaggtattctgttcaacccaaaaaattaccaagatttattagaacaaaatggccttgctccatggtataataaatttgaaagtttttatattcgtcgtatgaaacaaaaaattgatgattgttttgcaagaccaacttgtggtgtcccaggtagattaatcacttgtattgatcgtgatgctcatgattataattcatattttagttatcctggtactacttcaacttgtttaaatttatcatcatataattatttggggtttgcacaatctgaaggggcatgtactcaagccgctttagaaattttggattattatggtgttggttctggtggtccaagaaatgttattggtactactgatttacatttaaaaactgaaaaaactatagcaaaatttattggtaaagatgattcaatcttattttcaatggggtatgcaacaaatgcaagtttatttagttctttattggataagaaatcacttgttatttctgatgaattaaatcatgcttcaattagaactggtgttagattatctggttctacagttaaaactttccctcataataatatgattgccttggaaaaaattcttagagaacaaatttctcaaggtcaaccaagatctcatcgtccatggaaaaaaatcattgttgcagttgaagggctttattcaatggagggtacaatggcaaatttacctgcattaattgaattaagaagaaaatataaatttaatttatttgttgatgaagctcattcaattggtgctattggtccatcaggtcgtggtgtttgtgattattttggtatagatccctcaaatgttgatttattaatggggactttaactaaatcatttggtgctgcaggtggttatattgctggttcacaacaaattataaatcgtttaaaattaaatattaattcacaaaattatgcagaatctatccctgcacctgttttggcacaaattatttcttcgttaaatatcatctcgggtgatttaaatcctggtgaaggttcggaaagattagaaagaattgcttttaattcacgttatttaagattaggtttacaaagattaggttttatcgtatacggagttgatgattcaccagtgatcccattattattattcgccccagccaaaatgccagcattttcacgtatgctatatcaaggaaaaatttcagttgttgttgttggatacccggcaactccactgacttcatcaggagttcgtcttggtgttcctgcatctttaccaaaggaggatatggattatcttttacgtcatttatccgagttgggtgataaattatttttaaaatttagttctggtattgctggtggttctttagatggttcaccaccaagatggaatattgaagatgttttgaaagagactccaaaggattgtaaagaatctaaatattttattgcaactgcaaataattga

SEQ ID NO: 4 - protein, 349 aa, synthetic construct
  REGION 1..349: note = Amino acid sequence of Sphinganine C4-hydroxylase
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGYMYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 5 - DNA, 1017 bp, synthetic construct
  misc_feature 1..1017: note = polynucleotide sequence of SYR2
  source 1..1017: mol_type = other DNA
gatcatatacacaaaagacatgagctctcatcagtttttgatcaaccaaacaactttggcggctccacctgttcatttggtggagaaaccaagtttgattaatggcataccggataacattttagccttgattgcacctgttatagcttattattcatattcaggatttttctatgtgattgatactttagaaattgcagaactttatagaattcatccacctgaagaagttagttcaagaaataaagctacaaaatttgatgttttaaaagatgttgttttacaacattttatacagagtgttgttggttatatctttacatattttgatccaattcaatatactggtgatgaagaatatcaagcttggaaattacaacaaactttaccatttttaccatttgatgttgcatattattggaatatgtatggttggagttgtttgaaaattggtcttgcatttttaattattgattcatggcaatattggttacatagaattatgcatttaaacaagacattatacaaaagattccattcaagacatcatcgtctttatgtcccatatgcttttggtgctttatataatgatccatttgaagggtttttattggataccttaggtaccggtattgctgcaattgttactcaattaactccaagagaatctattgttttatatacattttcaactttgaaaactgttgatgatcattgtggttattcattaccttatgatcctttccaaattttgttcccaaataactcaatttatcatgatattcatcatcaacaatttggtatcaagaccaatttttcacaacctttctttacacattgggatgttttcagtaatacaagatataaagaaattgatgaatacagagaaaagcaaaaagctattacaattgccaaatataaagagtttttacatgatcgtgaaattgcaaaacaaaagaagaaggctgaaatttataaagataagaaaactgattgatttcgatcaaattttcatta

SEQ ID NO: 6 - protein, 624 aa, synthetic construct
  REGION 1..624: note = Amino acid sequence of long chain base kinase
MVVQKKLRAILTDEGVLIKSQSHHMFNKHGQLRSGDSLSLLSCLSCLDDGTLSSDGGSFDEDDSLELLPLNTTIPFNRILNAKYVNVGQKGFNNGKISSNPFQTENLSSSSENDDVENHSLSNDKAPVSESQSFPKKDKWDTKTNTVKVSPDDSQDNSPSLGIKDNQQLIELTFAVPKGHDVIPQKLTLLIDHVSRKSRANTGEENISSGTVEEILEKSYENSKRNRSILVIINPHGGKGTAKNLFLTKARPILVESGCKIEIAYTKYARHAIDIAKDLDISKYDTIACASGDGIPYEVINGLYRRPDRVDAFNKLAVTQLPCGSGNAMSISCHWTNNPSYAALCLVKSIETRIDLMCCSQPSYMNEWPRLSFLSQTYGVIAESDINTEFIRWMGPVRFNLGVAFNIIQGKKYPCEVFVKYAAKSKKELKVHFLENKDKNKGCLTFEPNPSPNSSPDLLSKNNINNSTKDELSPNFLNEDNFKLKYPMTEPVPRDWEKMDSELTDNLTIFYTGKMPYIAKDTKFFPAALPADGTIDLVITDARIPVTRMTPILLSLDKGSHVLEPEVIHSKILAYKIIPKVESGLFSVDGEKFPLEPLQVEIMPMLCKTLLRNGRYIDTEFESM

SEQ ID NO: 7 - DNA, 1875 bp, synthetic construct
  misc_feature 1..1875: note = polynucleotide sequence of LCB4
  source 1..1875: mol_type = other DNA
atggtggtgcagaaaaaacttagggctatcttgaccgatgaaggtgtattgatcaaatcgcaatcacaccatatgttcaataagcatggtcaactcagaagcggagattctttatccttgttgagctgcttgtcctgtctggatgatggaactttgagctctgatggaggttcttttgatgaggatgattccctggaactgttgcctcttaatactaccattccgttcaacagaattttgaacgcaaaatatgtgaatgtcggtcagaaaggcttcaataatggcaaaatttcttcgaatccttttcaaacggaaaatctgagttcttcgtctgaaaatgacgacgttgagaatcatagtttgagcaatgataaggctcctgtaagcgaatcacagtcatttcccaaaaaagacaagtgggatacaaaaacgaacactgtgaaggtgtctcccgatgattcacaggataactcaccatctttagggataaaagataatcaacagttaattgagttaacttttgctgtacccaagggccatgatgttataccacaaaaattaaccttgttaatagatcacgtttctaggaaatcgagagcaaataccggagaggagaacatttcttctggtactgtggaagaaatcctggaaaaaagttatgaaaattccaagagaaacagatcgatattagtcattattaatccccacggtggtaaaggtactgctaaaaatttattcctgacaaaagcaaggccaatactagtggaaagtggctgcaaaatagaaattgcatacacaaaatatgcccgtcacgccatcgatattgccaaagatttagatatcagcaaatacgataccattgcatgtgcctcgggtgatggtattccatacgaagtaattaatgggctttatagaagacccgacagagtggatgcgttcaataaactagccgtaactcagctaccttgcggttcaggaaatgctatgagcatttcatgtcattggacaaataacccatcgtacgccgctctgtgccttgtcaaatccattgaaacaagaatagacttaatgtgttgttcccaaccttcttatatgaacgaatggccaagattatcctttttgagtcagacgtacggcgttattgcagaatctgatattaacactgaattcatcagatggatgggtcccgttaggtttaatttgggtgtagcattcaacattatccaaggtaagaaatatccctgcgaagttttcgtcaaatatgctgccaaatctaaaaaggaattaaaagttcatttcttagaaaataaagataaaaacaaaggatgtttaaccttcgaaccaaatcctagcccaaactcttcgccggatttactatctaaaaacaatatcaacaacagtacaaaagatgaactttcaccgaattttctcaacgaggacaactttaaattaaagtatccgatgacggaaccagtacctagagactgggagaaaatggattcagagctaactgataacttaacaatcttttacacagggaaaatgccgtatattgctaaggacaccaaattttttcccgctgctttaccagcggatggtaccattgatttagtcataacggatgcaagaatcccagtgacaagaatgacaccaattttattatccttggataaaggttctcatgtattagagccagaagttattcactcaaaaatattggcttataagattataccaaaagtggagtcaggtttattttcagtggatggtgaaaagtttcctttggaacccttgcaagtggaaataatgcccatgttatgcaagacgttgctaaggaatggtagatatatcgatacagagtttgaatccatgtag

(19) United States
(12) Patent Application Publication
(10) Pub. No.: US 2026/0062724 A1
(43) Pub. Date: Mar. 5, 2026

(54) MUTANT WICKERHAMMYCES CIFERRll STRAIN HAVING IMPROVED PRODUCTIVITY FOR TAPS, AND METHOD FOR PRODUCING TAPS USING SAME

(71) Applicant: Korea University Research and Business Foundation, Seoul (KR)

(72) Inventors: Min-Kyu OH, Seongnam-si (KR); Seok Woo YOO, Yongin-si (KR)

(73) Assignee: Korea University Research and Business Foundation, Seoul (KR)

(21) Appl. No.: 19/147,244

(22) PCT Filed: Sep. 1, 2023
(86) PCT No.: PCT/KR2023/013073

§ 371 Date: Jul. 10, 2025

(30) Foreign Application Priority Data
Sep. 1, 2022 (KR) 10-2022-0110854

(51) Int. Cl. C12P 13/02; C12N 1/16; C12N 9/02; C12N 9/10; C12N 9/12; C12N 15/52; C12N 15/81; C12R 1/645

(52) U.S. Cl. CPC C12P 13/02; C12N 1/16; C12N 9/0073; C12N 9/1029; C12N 9/1205; C12N 15/52; C12N 15/815; C12Y 114/13169; C12Y 203/0105; C12Y 207/01091; C12N 2800/102; C12R 2001/645

(57) ABSTRACT

The present invention relates to a Wickerhamomyces ciferrii mutant strain having an improved ability to produce tetraacetylphytosphingosine (TAPS), and a method for producing TAPS using the same. The Wickerhamomyces ciferrii mutant strain provided by the present invention exhibits excellent TAPS productivity, and thus may be widely used in development of various products that use TAPS.

### TECHNICAL FIELD

The present invention relates to a Wickerhamomyces ciferrii mutant strain having an improved ability to produce TAPS and a method for producing TAPS using the same.

### BACKGROUND ART

Biorefinery is a technology that produces high value-added products such as biochemicals, biopolymers, and biofuels from biomass as a raw material. Biomass, an alternative to fossil fuels, refers to renewable organic materials including plants, animals, and microorganisms. Unlike limited fossil fuels, biomass is an infinite resource as long as solar energy exists. Therefore, biomass can be an alternative to reduce petroleum dependence and solve greenhouse gas problems. In recent years, the use of various platforms such as yeast, E. coli, Bacillus subtilis, and filamentous fungi has increased, among which yeast is the first eukaryotic organism whose genome has been sequenced and has a wide range of technology platforms in systems biology and synthetic biology.

Meanwhile, ceramide is the main component of the lipid layer in the stratum corneum (SC), the uppermost layer of the human skin epidermis. Ceramide is known as the most important skin lipid, accounting for 40 to 50% of lipids, and is used in cosmetics such as moisturizers and hand creams. Ceramide is a sphingolipid in which the acyl chain of a fatty acid is linked to a sphingoid base via an amide bond, and the sphingoid base has sphingosine, phytosphingosine, or sphinganine as a backbone. Sphingolipids are components that form the membrane structure of all living organisms and play a key role in cell growth and proliferation. Tetraacetyl phytosphingosine (TAPS) is a fully acetylated form of phytosphingosine. In particular, TAPS can be converted to phytosphingosine, one of the sphingoid bases of ceramides, through a process of removing the acetyl group, and therefore has been reported to be important for ceramide synthesis. TAPS synthesis starts with the condensation of L-serine and palmitoyl-CoA by catalyzed by serine palmitoyltransferase (SPT), resulting in the synthesis of 3-keto-sphinganine, which is then converted to sphinganine and phytosphingosine (see FIG. 1). Phytosphingosine has a D-erythro (2S, 3R) configuration which is the same as that of sphingosine present in human skin. In addition, it has been reported that phytosphingosine is effective for skin diseases accompanied by chronic inflammation, such as atopy, because it penetrates deep into the skin and promotes ceramide synthesis.

Pichia ciferrii, previously known to have the property of producing TAPS and secreting the same to the outside of the cell, is an unconventional yeast having properties different from those of the conventional Saccharomyces cerevisiae yeast. Many studies have been conducted to improve TAPS production in Wickerhamomyces ciferrii (W. ciferrii), and a typical method is to construct strains with improved TAPS production either by the introduction of random mutations using mainly ethyl methane sulfonate (EMS), γ-ray irradiation, UV and NTG, or through genetic engineering.

Thereamong, the conventional method of genetically engineering Wickerhamomyces ciferrii is a genetic engineering method based on homologous recombination, and in particular, promoter replacement or gene insertion was performed for overexpression of a specific gene. However, there were several disadvantages, such as much time and labor required to remove the selection marker, and the traces remaining in the gene. Therefore, it is still necessary to establish an efficient genetic tool for overexpression.

### DISCLOSURE

### Technical Problem

An object of the present invention is to provide a novel expression plasmid for genetic engineering of Wickerhamomyces ciferrii.

Another object of the present invention is to provide a Wickerhamomyces ciferrii mutant strain having an improved ability to produce tetraacetylphytosphingosine.

Still another object of the present invention is to provide a composition for producing tetraacetylphytosphingosine, comprising the mutant strain.

Yet another object of the present invention is to provide a method of producing tetraacetylphytosphingosine using the Wickerhamomyces ciferrii mutant strain.

Still yet another object of the present invention is to provide a method for constructing the Wickerhamomyces ciferrii mutant strain.

### Technical Solution

Each description and embodiment disclosed in the present invention may also be applied to each other description and embodiment. That is, all combinations of various elements disclosed in the present invention fall within the scope of the present invention. In addition, the scope of the present invention may not be considered to be limited by the specific description described below.

Furthermore, those skilled in the art will recognize, or be able to ascertain, using no more than routine experimentation, numerous equivalents to the specific embodiments described herein. Such equivalents are also intended to be encompassed by the present invention.

In addition, throughout the present specification, it is to be understood that when any part is referred to as “comprising” any component, it does not exclude other components, but may further comprise other components, unless otherwise specified.

The present invention has been made in order to solve the problem of difficulty in applying expression plasmids to conventional Wickerhamomyces ciferrii strains, and is based on construction of an expression plasmid for transformation into Wickerhamomyces ciferrii and on the new discovery that genetic engineering using the expression plasmid can more effectively improve TAPS production.

Hereinafter, preferred embodiments of the present invention will be described in detail with reference to the accompanying drawings.

To achieve the above objects, the present invention provides an expression plasmid for transformation into a Wickerhamomyces ciferrii strain.

Specifically, the expression plasmid is characterized by containing a CEN/ARS origin of replication and a uracil selection marker.

The term “Wickerhamomyces ciferrii strain” as used in the present invention refers to a yeast strain that produces various sphingolipids including TAPS. TAPS is known to be biosynthesized from serine and palmitoyl-CoA by a chain reaction in the strain. More specifically, first, the condensation reaction of serine and palmitoyl-CoA catalyzed by serine C-palmitoyltransferase encoded by the LCB1 and LCB2 genes occurs, synthesizing 3-keto-sphingonine. Second, 3-keto-sphingonine is reduced to 3-keto-sphinganine by 3-keto-sphinganine reductase encoded by the TSC10 gene. Third, 3-keto-sphinganine is hydroxylated into phytosphingosine by sphinganine C-4-hydroxylase encoded by the SYR2 gene. Finally, The N and O moieties of phytosphingosine are acetylated by acetyltransferases encoded by the Sl1i1 and Atf2 genes, thereby producing TAPS (FIG. 1).

The term “plasmid” as used in the present invention refers to a representative episomal DNA molecule physically separated from the chromosomal DNA essential for growth. The term “plasmid” was first introduced by American geneticist J. Lederberg in 1952 to describe a bacterial extrachromosomal DNA molecule capable of independent proliferation. Plasmids exist in various sizes, from 1 kilobase to 1 megabase, and are mostly circular, but linear plasmids have also been reported. Plasmids can be isolated from bacteria and transferred into new host cells, and plasmids that can be easily transformed are widely used in genetic engineering. A vector obtained by extracting DNA from bacteria and containing multiple restriction enzyme cloning sites is used to promote replication of recombinant DNA sequences. In addition, an expression vector is also used to express the gene of interest as a protein within a cell.

As used in the present invention, the term “origin of replication” refers to a particular DNA sequence in a genome at which DNA replication is initiated, and is also called the replication origin. In prokaryotes, DNA replication begins from a single origin of replication and proceeds bidirectionally. The origins of replication are mostly characterized by repeated A and T nucleotides, and because they have a high content of A and T, the double helix structure is more easily unraveled than other regions of DNA.

The present invention is characterized in that the replication origin is a CEN/ARS origin of replication.

In the CEN/ARS, CEN is an abbreviation for centromere, and ARS is an abbreviation for autonomous replicating sequence, and is a region that characterizes the type of yeast gene transporter.

As used in the present invention, the term “selection marker” refers to one used to confirm whether the plasmid has been properly introduced into the transformant, and is also called a selectable marker. The present invention is characterized by using a uracil selection marker to confirm whether the expression plasmid has been properly introduced into the Wickerhamomyces ciferrii strain by transformation.

The uracil is a pyrimidine base derivative and exists in RNA but not in DNA. In the present invention, an example of the uracil selection marker may be 5-fluoroorotic acid (5-FOA) monohydrate, without being limited thereto.

5-Fluoroorotic acid (5-FOA) is used in yeast genetics to confirm the absence of the URA3 gene, because URA3 encodes the enzyme that catalyzes the decarboxylation of 5-FOA to the toxic metabolite 5-fluorouracil, leading to death.

In a specific example of the present invention, a uracil auxotrophic strain was constructed using 5-FOA during strain screening. In addition, it was confirmed that the constructed uracil auxotrophic strain contained 5-FOA as a uracil selection marker in the plasmid.

The expression plasmid of the present invention may further contain a gene encoding serine palmitoyl-transferase and a gene encoding sphinganine C4-hydroxylase.

As used in the present invention, the term “serine palmitoyl-transferase” refers to a protein involved in producing 3-ketosphinganine from L-serine and palmitoyl-CoA.

The protein may be the amino acid sequence of SEQ ID NO: 1 and/or an amino acid sequence having at least 70%, 80%, 85%, 90%, 95%, 96%, 97%, 98%, or 99% homology or identity to SEQ ID NO: 1. In addition, it is obvious that an auxiliary protein having an amino acid sequence having deletion, modification, substitution or addition in a portion of the sequence is also included within the scope of the present application, as long as it has such homology or identity and exhibits an effect corresponding to that of the protein.

In addition, a probe that may be prepared from a known gene sequence, for example, a polypeptide having serine palmitoyl-transferase activity, which is a polypeptide encoded by a polynucleotide that hybridizes under stringent conditions with a sequence complementary to all or part of the nucleotide sequence encoding the polypeptide, may also be included without limitation.

The serine palmitoyl-transferase protein may be encoded by an LCB1 polynucleotide, an LCB2 polynucleotide, or a combination thereof.

As used in the present invention, the term “polynucleotide” is meant to comprehensively include DNA or RNA molecules, and nucleotides, which are the basic structural units in polynucleotides, may include not only natural nucleotides but also analogs in which sugar or base moieties are modified (see Scheit, Nucleotide Analogs, John Wiley, New York (1980); Uhlman and Peyman, Chemical Reviews, 90:543-584 (1990)).

A polynucleotide encoding the protein comprising the amino acid sequence of SEQ ID NO: 1 may include, without limitation, any sequence capable of encoding a protein having the activity of a serine palmitoyl-transferase protein or a functional fragment thereof.

For example, the LCB1 gene may comprise the polynucleotide of SEQ ID NO: 2, and the LCB2 may comprise the polynucleotide of SEQ ID NO: 3.

In addition, it is obvious that the present invention may also include a sequence obtained by codon-optimizing the gene sequence based on the codon frequency of the parent strain.

As used in the present invention, the term “sphinganine C4-hydroxylase” refers to a protein involved in the conversion of sphinganine to phytosphingosine.

The protein may be the amino acid sequence of SEQ ID NO: 4 and/or an amino acid sequence having at least 70%, 80%, 85%, 90%, 95%, 96%, 97%, 98%, or 99% homology or identity to SEQ ID NO: 4. In addition, it is obvious that an auxiliary protein having an amino acid sequence having deletion, modification, substitution or addition in a portion of the sequence is also included within the scope of the present application, as long as it has such homology or identity and exhibits an effect corresponding to that of the protein.

The sphinganine C4-hydroxylase may be encoded by SYR2. The SYR2 gene may comprise, for example, the polynucleotide of SEQ ID NO: 5.

The expression plasmid of the present invention may further contain homology arms of a gene encoding a long-chain base kinase.

As used in the present invention, the term “long-chain base kinase” refers to a protein that converts sphinganine into sphinganine-1-P and may comprise the amino acid sequence of SEQ ID NO: 6.

The protein may be encoded by an LCB4 polynucleotide sequence, and for example, the gene LCB4 sequence may comprise the polynucleotide of SEQ ID NO: 7.

Homology in the term “homology arms” as used in the present invention refers to the preservation of certain characteristics during the course of evolution, and this can apply to morphological characteristics, molecular characteristics, and gene sequences. Therefore, the homology arms may be referred to as homologous nucleotide sequences.

In one embodiment, the expression plasmid of the present invention may be an expression plasmid containing a CEN/ARS origin of replication origin and/or a uracil selection marker, and further containing a gene encoding serine palmitoyl-transferase and a gene encoding sphinganine C4-hydroxylase. For example, the expression plasmid, which comprises a p416GPD plasmid as a backbone and the CEN/ARS origin of replication and overexpresses the genes LCB1 and LCB2 encoding serine palmitoyl-transferase and the gene SYR2 encoding sphinganine C4-hydroxylase, may be pEXP, without being limited thereto.

In another embodiment, the expression plasmid of the present invention may be an expression plasmid further containing homology arms of a gene encoding a long-chain base kinase. As an example, the expression plasmid may be pCM1842, a deletion plasmid comprising a pCM184 plasmid as a backbone, a uracil selection marker, and homology arms of LCB4, a gene encoding a long-chain base kinase, which may be deleted by the cre-loxP system, without being limited thereto.

The vector of the present invention may direct the expression of an operably linked gene encoding the protein of interest, and such a vector is called an expression vector. Generally, in the use of recombinant DNA technology, the expression vector is in the form of a plasmid.

The type of expression vector that may be used may be determined depending on the type of host cell. When Wickerhamomyces ciferrii is used as the host, for example, a pSH471, pSH472, pEXP, pCM1841, or pCM1842 vector may be used as the expression vector, and an ENO1 promoter, TDH3 promoter, or GPD promoter may be used as the promoter, without being limited thereto.

As a method for introducing DNA into Wickerhamomyces ciferrii, for example, an electroporation method (Method Enzymol., 194, 182-187(1990)), a spheroplast method (Proc. Natl. Acad. Sci. USA, 84, 1929-1933(1978)), a lithium acetate method (J. Bacteriol., 153, 163-168(1983)), or the like may be used.

In addition, the expression vector may further contain silencing fragments having various functions of inhibiting, amplifying or inducing the expression of the gene of interest, a marker for selecting a transformant, an antibiotic resistance gene, a gene encoding a signal for extracellular secretion, a fusion partner suitable for a poorly expressed protein, or the like.

As used in the present invention, the term “transformation” means introducing DNA into a host cell so that the DNA becomes replicable as an extrachromosomal factor or by chromosomal integration.

Any host cell widely known in the art may be used for the transformation according to the present invention. However, a host having high efficiency of introduction and expression of the genes encoding LCB1, LCB2 and SYR2 of the present invention may be used, and further, the LCB4 gene may be deleted therefrom. The present invention is characterized by using Wickerhamomyces ciferrii.

Another embodiment of the present invention for achieving the above objects provides a Wickerhamomyces ciferrii strain having an improved ability to produce tetraacetylphytosphingosine (TAPS), obtained by further enhancing the activities of serine palmitoyl-transferase and sphinganine C4-hydroxylase and further weakening the activity of a long-chain base kinase using the expression plasmid containing the CEN/ARS origin of replication and the uracil selection marker.

As used in the present invention, the terms “Wickerhamomyces ciferrii strain”, “CEN/ARS origin of replication”, “uracil selection marker”, “expression plasmid”, “serine palmitoyl-transferase”, “sphinganine C4-hydroxylase”, and “long-chain base kinase” are as described above.

The term “tetraacetylphytosphingosine (TAPS)” as used in the present invention means a compound having a structure of Formula I below.

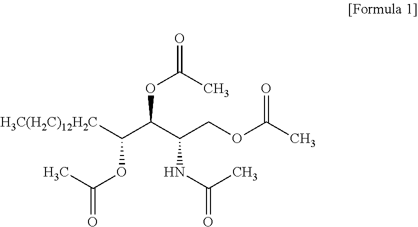

For the biosynthesis of TAPS in the microorganism of the present invention, it is known that TAPS is biosynthesized through a series of biosynthetic processes shown in FIG. 1.

It was confirmed that the Wickerhamomyces ciferrii strain provided in the present invention is a novel Wickerhamomyces ciferrii mutant strain having an improved ability to produce TAPS due to an improved metabolic flux for synthesizing palmitoyl-CoA. In the present invention, the term “Wickerhamomyces ciferrii strain” may be used interchangeably with the term “Wickerhamomyces ciferrii mutant strain”.

In addition, the novel Wickerhamomyces ciferrii strain of the present invention may be one deposited under the accession number KCTC14970BP.

As used in the present invention, the term “activity . . . is enhanced” or “activity . . . is weakened” refers to enhancing or weakening the activity of a protein, respectively.

The method of introducing, enhancing, or inactivating the activity of a specific protein and/or gene may be performed using a suitable method known in the art depending on the characteristics of the microorganism.

As used in the present application, the term “enhancing” the activity of a protein means that the activity of the protein is introduced or increased compared to the intrinsic activity. The term “introducing” the activity means that the activity of a specific polypeptide that is not originally possessed by a microorganism is naturally or artificially exhibited.

As used in the present application, the term “increase” in the activity of a protein compared to the intrinsic activity means that the activity is increased compared to the intrinsic activity of a protein possessed by the microorganism or the activity of the protein before modification. The “intrinsic activity” refers to the activity of a specific protein originally possessed by a parent strain or unmodified microorganism before the characteristics of the microorganism is changed by genetic mutation due to natural or artificial factors. The term “intrinsic activity” may be used interchangeably with the term “activity before modification”. The increase in activity may include both introducing a foreign protein and enhancing the activity of an endogenous protein. Increasing/enhancing the activity of the protein may be achieved by increasing/enhancing the expression of the gene.

Specifically, in the present application, increasing the activity may be performed by, but is not limited to:
1) increasing the copy number of a polynucleotide encoding the protein;
2) modifying an expression regulatory sequence to increase the expression of the polynucleotide;
3) modifying the polynucleotide sequence on the chromosome to enhance the activity of the protein;
4) introducing a foreign polynucleotide exhibiting the activity of the protein or a mutant polynucleotide obtained by codon-optimizing the polynucleotide; or
5) a method of inducing modification by a combination thereof to enhance the activity.

1) Increasing the copy number of a polynucleotide may be performed in a form operably linked to a vector, or by insertion into a chromosome in a host cell, without being particularly limited thereto. Specifically, the increasing the copy number may be performed by introducing into a host cell a polynucleotide encoding the protein of the present invention, which is operably linked to a vector capable of replicating and functioning independently of the host, or by introducing into a host cell the polynucleotide operably linked to a vector capable of inserting the polynucleotide into a chromosome in the host cell, thereby increasing the copy number of the polynucleotide in the chromosome of the host cell.

Next, 2) modifying an expression regulatory sequence to increase the expression of the polynucleotide may be performed by, but is not particularly limited to, inducing a mutation in the nucleic acid sequence by deletion, insertion, non-conservative or conservative substitution, or a combination thereof to further enhance the activity of the expression regulatory sequence, or by replacing the nucleic acid sequence with a nucleic acid sequence having stronger activity. The expression regulatory sequence may include, but is not particularly limited to, a promoter, an operator sequence, a sequence encoding a ribosome binding site, a sequence for regulating the termination of transcription and translation, etc.

A strong heterologous promoter may be linked upstream of the polynucleotide expression unit instead of the original promoter, but is not limited thereto. In addition, 3) modifying the polynucleotide sequence on the chromosome may be performed, but is not particularly limited thereto, by inducing a mutation in the expression regulatory sequence by deletion, insertion, non-conservative or conservative substitution, or a combination thereof of a nucleic acid sequence to further enhance the activity of the polynucleotide sequence, or by replacement with a polynucleotide sequence improved to have stronger activity.

In addition, 4) introducing a foreign polynucleotide sequence may be performed by introducing a foreign polynucleotide encoding a protein exhibiting an activity identical to/similar to that of the protein, or a mutant polynucleotide obtained by codon-optimization thereof, into the host cell. The foreign polynucleotide may be used without limitation in its origin or sequence as long as it exhibits an activity identical to/similar to that of the protein. In addition, the foreign polynucleotide may be introduced into the host cell after optimizing its codon so that optimized transcription and translation may occur within the host cell. The introduction may be performed using a known transformation method appropriately selected by a person skilled in the art, and the introduced polynucleotide may be expressed within the host cell, thereby producing a protein with increased activity.

Finally, 5) the method of inducing modification by a combination of 1 to 4) above to enhance the activity may be performed by applying at least one of the following methods: increasing the copy number of a polynucleotide encoding the protein; modifying an expression regulatory sequence to increase the expression of the polynucleotide; modifying the polynucleotide sequence on the chromosome; and introducing a foreign polynucleotide exhibiting the activity of the protein or a mutant polynucleotide obtained by codon optimization thereof.

In a specific example of the present invention, an effective expression plasmid that can work in Wickerhamomyces ciferrii was constructed, the activities of protein serine palmitoyl transferase and sphinganine C-4-hydroxylase were enhanced by introducing LCB1, LCB2 and SYR2 genes in a simple and effective manner by using this specific expression plasmid, and furthermore, the long-chain base kinase activity was weakened by deleting the LCB4 gene. As a result, it was confirmed that the produced Wickerhamomyces ciferrii strain had a significantly increased ability to produce TAPS.

Another embodiment of the present invention for achieving the above objects provides a Wickerhamomyces ciferrii strain having an improved ability to produce tetraacetylphytosphingosine (TAPS), constructed by enhancing the activities of serine palmitoyl-transferase and sphinganine C4-hydroxylase and further weakening the activity of long-chain base kinase in a uracil auxotrophic Wickerhamomyces ciferrii strain containing a uracil selection marker by using an expression plasmid containing a CEN/ARS origin of replication.

As used herein, the terms “Wickerhamomyces ciferrii strain”, “CEN/ARS origin of replication”, “uracil selection marker”, “expression plasmid”, “serine palmitoyl-transferase”, “sphinganine C4-hydroxylase”, “long-chain base kinase” and “tetraacetylphytosphingosine (TAPS)” are as described above.

Still another embodiment of the present invention provides a composition for producing tetraacetylphytosphingosine (TAPS), comprising the strain, a culture of the strain, a lysate of the strain, or an extract of the strain.

As used in the present invention, the terms “Wickerhamomyces ciferrii strain” and “tetraacetylphytosphingosine (TAPS)” are as described above.

As used in the present invention, the term “culture of the strain” means a culture of the Wickerhamomyces ciferrii strain of the present invention, and refers to a culture product obtained by culturing the strain of the present invention in a medium. For example, the culture of the strain may include a filtrate of a culture of the strain of the present invention or a culture supernatant obtained by centrifuging the culture.

The term “lysate thereof” means a lysate of the Wickerhamomyces ciferrii strain of the present invention, and is meant to include all useful substances present in the strain, which may be released by lysing the strain.

The term “extract thereof” means an extract obtained by extracting the Wickerhamomyces ciferrii strain of the present invention using a solvent. As the solvent, any known solvent may be used without limitation, and any extraction method may also be used without limitation.

The term “extract of the culture” means an extract obtained by extracting a culture of the Wickerhamomyces ciferrii strain of the present invention using a solvent. As the solvent, any known solvent may be used without limitation, and any extraction method may also be used without limitation.

The term “extract of the lysate” means an extract obtained by extracting a lysate of the Wickerhamomyces ciferrii strain of the present invention using a solvent. As the solvent, any known solvent may be used without limitation, and any extraction method may also be used without limitation.

The Wickerhamomyces ciferrii strain of the present invention, a culture thereof, a lysate thereof, an extract thereof, an extract of a culture thereof, or an extract of the lysate may be comprised in an amount of 10 to 50 wt % based on the weight of the composition, without being limited thereto. Specifically, if it is comprised in an amount of less than 10 wt %, the effect will be insufficient, and if it is comprised in an amount of more than 50 wt %, the increase in effect for the amount used will be insufficient, which may be uneconomical.

Yet another embodiment of the present invention provides a method for producing tetraacetylphytosphingosine using the Wickerhamomyces ciferrii mutant strain.

Specifically, the method for producing tetraacetylphytosphingosine according to the present invention comprises a step of culturing the above-described Wickerhamomyces ciferrii mutant strain to obtain a culture, and may further comprise a step of recovering tetraacetylphytosphingosine from the obtained culture.

Here, the terms “Wickerhamomyces ciferrii” and “tetraacetylphytosphingosine” are as described above.

The term “culturing” refers to allowing the recombinant microalgae to grow, and the medium and culture conditions for culturing the transformant of the present invention may be appropriately selected and used depending on the host cell. The nutrient medium preferably contains a carbon source, inorganic nitrogen source, or organic nitrogen source necessary for the growth of the host cell. Examples of the carbon sources include glucose, dextran, soluble starch, sucrose, and methanol. Examples of the inorganic or organic nitrogen sources include ammonium salts, nitrates, amino acids, corn steep liquor, peptone, casein, bovine extract, soybean white, and potato extract. If necessary, the nutrient medium may contain other nutrients, for example, inorganic salts such as sodium chloride, calcium chloride, sodium dihydrogen phosphate, and magnesium chloride, vitamins, and antibiotics (tetracycline, neomycin, ampicillin, and kanamycin). During culturing, conditions such as temperature, pH of the medium, and culturing time may be appropriately adjusted to suit cell growth and mass production of proteins.

In the above method, the step of culturing the microorganism is not particularly limited thereto, but may be performed by a known batch culture method, continuous culture method, fed-batch culture method, or the like. As an example, the microorganism may be cultured by a fed-batch culture method.

Here, the culture conditions are not particularly limited, but may be adjusted to an appropriate pH (e.g., pH 5 to 9, specifically pH 6 to 8, most specifically pH 6.8) using a basic compound (e.g., sodium hydroxide, potassium hydroxide, or ammonia) or an acidic compound (e.g., phosphoric acid or sulfuric acid), and oxygen or an oxygen-containing gas mixture may be introduced into the culture medium to maintain aerobic conditions. The culturing temperature may be maintained at 20 to 45° C., specifically 25 to 40° C., and the culturing may be performed for about 10 to 200 hours, without being limited thereto. The putrescine produced by the culturing may be secreted into the medium or remain within the cells.

In addition, the culture medium used may contain serine, glutamate, glycine, or a combination thereof, and specifically may contain a combination of glutamate and glycine.

In a specific embodiment of the present invention, it was confirmed that, when the novel Wickerhamomyces ciferrii strain of the present invention was fed-batch cultured, TAPS was effectively produced and the yield was also higher when a combination of glutamate and glycine was added to the medium compared to when other amino acids or one amino acid was added.

The culture medium may further contain a carbon source, a nitrogen source, and/or a phosphorus source, and may further contain other sources.

Examples of the carbon source include, but are not limited to, sugars and carbohydrates (e.g., glucose, sucrose, lactose, fructose, maltose, molasses, starch, and cellulose), fats and oils (e.g., soybean oil, sunflower seed oil, peanut oil, and coconut oil), fatty acids (e.g., palmitic acid, stearic acid, and linoleic acid), alcohols (e.g., glycerol and ethanol), and organic acids (e.g., acetic acid), which may be used alone or in combination. Examples of the nitrogen source include, but are not limited to, nitrogen-containing organic compounds (e.g., peptone, yeast extract, meat juice, malt extract, corn steep liquor, soybean meal, and urea), or inorganic compounds (e.g., ammonium sulfate, ammonium chloride, ammonium phosphate, ammonium carbonate, and ammonium nitrate), which may be used alone or in combination.

Examples of the phosphorus source include, but are not limited to, potassium dihydrogen phosphate, dipotassium hydrogen phosphate, and corresponding sodium-containing salts, which may be used alone or in combination. For example, the phosphorus source may be KH₂PO₄.

In addition, the medium may contain essential growth-promoting substances such as other metal salts (e.g., magnesium sulfate or iron sulfate), amino acids, and vitamins.

In addition, the medium may further containing a defoaming agent.

As used in the present invention, the term “defoaming agent” refers to an agent that removes harmful foam. Generally, an oily substance with low volatility and high diffusivity, or a water-soluble surfactant may be used.

The method for recovering TAPS produced in the culturing step of the present invention may comprise collecting the desired amino acid from the culture using a suitable method known in the art selected depending on the culturing method. For example, centrifugation, filtration, anion exchange chromatography, crystallization, and HPLC may be used, and the desired TAPS may be recovered from the medium or the microorganism using a suitable method known in the art.

Still yet another embodiment of the present invention provides a method for constructing a Wickerhamomyces ciferrii strain having an improved ability to produce tetraacetylphytosphingosine (TAPS). Specifically, the method may comprise: constructing a uracil auxotrophic Wickerhamomyces ciferrii strain; enhancing the activities of serine palmitoyl-transferase and sphinganine C4-hydroxylase using an expression plasmid containing a CEN/ARS origin of replication; and weakening the activity of long-chain base kinase.

Here, the descriptions of “Wickerhamomyces ciferrii”, “tetraacetylphytosphingosine”, “CEN/ARS origin of replication”, “expression plasmid”, “serine palmitoyl-transferase”, “sphinganine C4-hydroxylase”, “enhancing the activity”, “long-chain base kinase” and “weakening the activity” are as described above.

In a specific example of the present invention, in order to express a gene in a simple and effective manner by constructing an effective expression plasmid that can work in Wickerhamomyces ciferrii, various replication origin tests previously used in yeast were performed to find a CEN/ARS origin, and in order to obtain an efficient selection marker, a uracil auxotrophic Wickerhamomyces ciferrii strain was constructed using 5-FOA. Next, the gene to be overexpressed was cloned into an expression plasmid containing the CEN/ARS origin and the uracil selection marker, and then the expression plasmid was transformed into the constructed uracil auxotrophic Wickerhamomyces ciferrii strain, and the strain YP1 with the highest TAPS production was selected. It was confirmed that the final titer of YP1 was 2.68 g/L, which was 1.8 times higher than the titer of the uracil auxotrophic strain, 1.46 g/L. In addition, it was confirmed that the TAPS titer of YPD7 obtained by deleting the LCB4 gene was 3.03 g/L, which was 2 times higher than that of the control group. Finally, as a result of optimizing the medium conditions by adding amino acids, which are precursors involved in serine synthesis, to the medium, it was confirmed that the TAPS titer in the final strain YPD7 was 20 g/L, indicating that TAPS could be produced at a much higher concentration than previously known.

This suggests that, when a uracil auxotrophic Wickerhamomyces ciferrii mutant strain constructed using an expression plasmid having a specific origin of replication and a uracil selection marker is engineered, the TAPS production in fed-batch culture of the strain may be more effectively increased.

### Advantageous Effects

The Wickerhamomyces ciferrii mutant strain provided in the present invention has excellent TAPS productivity, and thus may be widely used in the development of various products using TAPS.

### BRIEF DESCRIPTION OF DRAWINGS

FIG. 1 shows the biosynthetic pathway of TAPS.

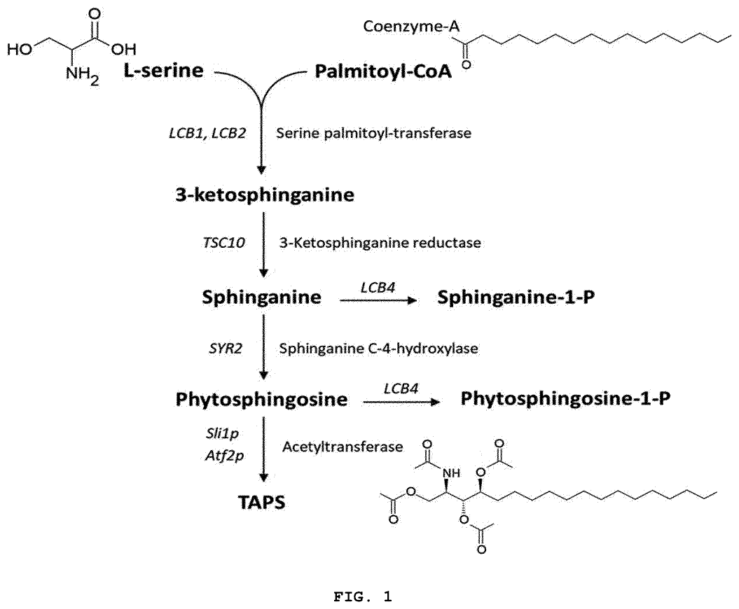

FIG. 2 shows a schematic view of the present invention.

FIG. 3 shows the process of constructing uracil auxotrophic Wickerhamomyces ciferrii.

FIG. 4 shows the ends-in and ends-out methods in the cre-loxP system.

FIG. 5 is a diagram showing four types of plasmids that are used for yeast.

FIG. 6 shows a CEN/ARS origin with KanMX and URA3 selection markers.

FIG. 7 shows EGFP expression using a CEN/ARS-derived expression plasmid.

FIG. 8 shows TAPS production following amino acid supplementation.

FIG. 9 shows a candidate Y94 strain with the expression plasmid pEXP.

FIG. 10 shows the TAPS titers and yields of the YP1 strain and the YP11 strain.

FIG. 11 shows the TAPS titers and yields of the YD94 strain and the YPD7 strain.

FIG. 12 shows high-cell-density fermentation using the final strain YPD7.

### BEST MODE

Hereinafter, the present invention will be described in more detail through examples. These examples are intended to explain the present invention in more detail, and the scope of the present invention is not limited by these examples.

### Experimental Example 1: Construction of Strains and Plasmids

Considering that expression plasmids have not yet been applied to Wickerhamomyces ciferrii, an expression plasmid that can work in Wickerhamomyces ciferrii was constructed to establish a simpler and more effective gene overexpression method.

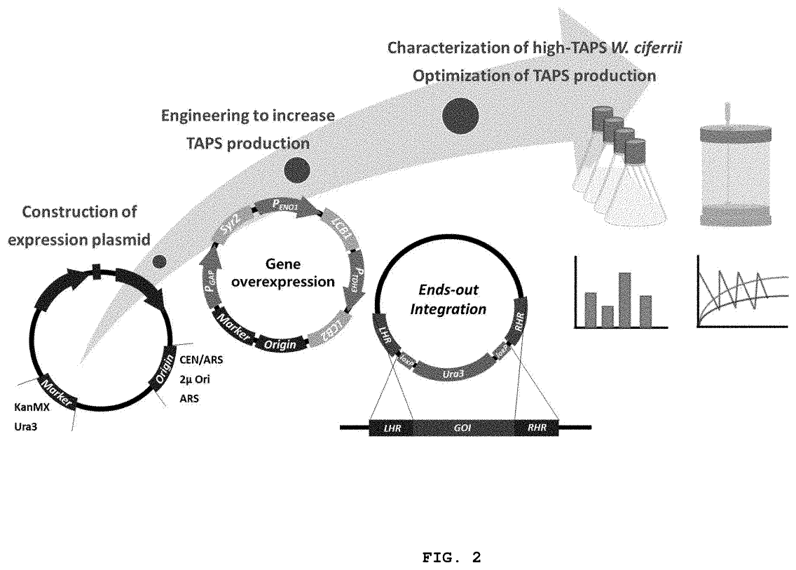

First, a uracil auxotrophic strain was constructed by optimizing the origin of replication and selection marker in an expression plasmid. Then, LCB1 and LCB2 encoding SPT and SYR2 encoding sphinganine C4-hydroxylase were overexpressed to increase TAPS production. In addition, LCB4 was deleted to improve TAPS production. Finally, high-cell-density fermentation was performed to achieve high production of TAPS (FIG. 2).

All engineered strains, recombinant plasmids and oligomers used in the present invention are shown in Table 1 and Table 2 below.

TABLE 1

| Name | Description | Reference |
|---|---|---|
| Strains | | |

WT

| | | |
|---|---|---|
| Y94 | W. ciferrii uracil auxotroph | This study |
| YP1 | Y94 with pEXP, colony No. 1 | This study |
| YD94-M | Y94 ΔLCB4 with selection marker | This study |
| YD94 | Y94 ΔLCB4 without selection marker | This study |
| YPD7 | Y94 ΔLCB4 with pEXP, colony No. 7 | This study |
| Plasmids | | |
| pSH47 | AmpR, ColE1 origin, GAL1 promoter, | Addgene |
| | Cre recombinase::URA3, CEN/ARS | |
| | origin | |
| pSH471 | pSH47::egfp | This study |
| pSH471_ARS | pSH471::URA3, ARS origin | This study |
| | ΔCEN/ARS | |
| pSH471_2μ | pSH471::URA3, 2μ origin | This study |
| | ΔCEN/ARS | |
| pSH472 | pSH471::KanMX ΔURA3, | This study |
| pSH472_ARS | pSH472::KanMX, ARS origin | This study |
| | ΔCEN/ARS | |
| pSH472_2μ | pSH472::KanMX, 2μ origin | This study |
| | ΔCEN/ARS | |
| p416GPD | AmpR, CEN/ARS origin, pBR322 | ATCC |
| | origin, GPD promoter, URA3 | (Manassas, |
| | | USA) |
| pEXP | p416GPD harboring P_{ENO1}::LCB1, | This study |
| | P_{TDH3}::LCB2, P_{GPD}::SYR2 | |
| pCM184 | AmpR, KanR, ColE1 origin, loxP | Addgene |
| pCM1841 | pCM184::URA3 | This study |
| pCM1842 | pCM1841::homology arms of LCB4 | This study |
| | from W. ciferrii | |

TABLE 2

| Oligomer | Sequence (5′→3′) | Description |
|---|---|---|
| egfp_for | tggatcccccgggctgcaggatctcgccac | Cloning for egfp |
| | CATGGTGAGCAAGGGCGAG | |
| egfp_rev | gcccctcgacggtatcgataTTACTTG | |
| | TACAGCTCGTCCATG | |
| KanMX_for | ctttaatttgcggccggtacCAGTATAG | Cloning for |
| | CGACCAGCATTC | KanMX |
| KanMX_rev | gcacagaacaaaaacctgcaGACATG | |
| | GAGGCCCAGAATAC | |
| ARS_for | tgcctgtaacttTCCCTTGTTTGAT | Cloning for ARS |
| | TCAGAAG | |
| ARS_rev | gggttccgcgcacatttccccgaaaACTA | |
| | TGTGTTGCCCTACC | |
| 2μ_for | cgcacatttccccgaaaagtGCCTCGT | Cloning for 2μ |
| | GATACGCCTATTTTTATAG | |
| 2μ_rev | aggttttcaccgtcatcaccAAAGTGC | |
| | CACCTGAACGAAG | |
| SYR2_for | GATCATATACACAAAAGAC | Amplification of |
| SYR2_rev | TAATGAAAATTTGATCGAA | SYR2 |
| | A | |
| ENO1_for | CAGATCAAACCACATCATG | Amplification of |
| ENO1_rev | TGTGTAATGTGTATATGTTT | ENO1 |
| | TATC | |
| LCB1_for | ATGAACGTCACTGCTACAA | Amplification of |
| | C | LCB1 |
| LCB1_rev | TTAAATAACTTCTTCAGTTA | |
| | ATAATGATAATG | |
| TDH3_for | GGACCGTTAATTACCAAC | Amplification of |
| TDH3_rev | TGTTAATTAATTATTTGTTTG | TDH3 |
| | TTTGTTTG | |
| LCB2_for | ATGTCATTGGTAATACCTCA | Amplification of |
| | AATAG | LCB2 |
| LCB2_rev | TCAATTATTTGCAGTTGCAA | |
| | TAAAATATTTAG | |
| EO_for | cgaagttatctagacctgcaCGGTGAA | Cloning for URA3 |
| | AACCTCTGACAC | |
| EO_rev | gaagttatctaggacctgcaTTTCACA | |
| | CCGCATAGGGTAATAAC | |
| LCB4_LHR_ | ATATATAGAATTCTGCGCTAT | Cloning for LHR of |
| for | GATTATTTAAGGACTTT | LCB4 |
| LCB4_LHR_ | ATATATGGTACCTGGATGGG | |
| rev | TTGAAGTATGTCTTT | |
| LCB4_RHR_ | cgcgtgttaaccggtgagctTTGAAAT | Cloning for RHR |
| for | TCTACCCGGTG | of LCB4 |
| LCB4_RHR_ | gctggatcctctagtgagctACACCAG | |
| rev | TAGCTGCATTTTG | |

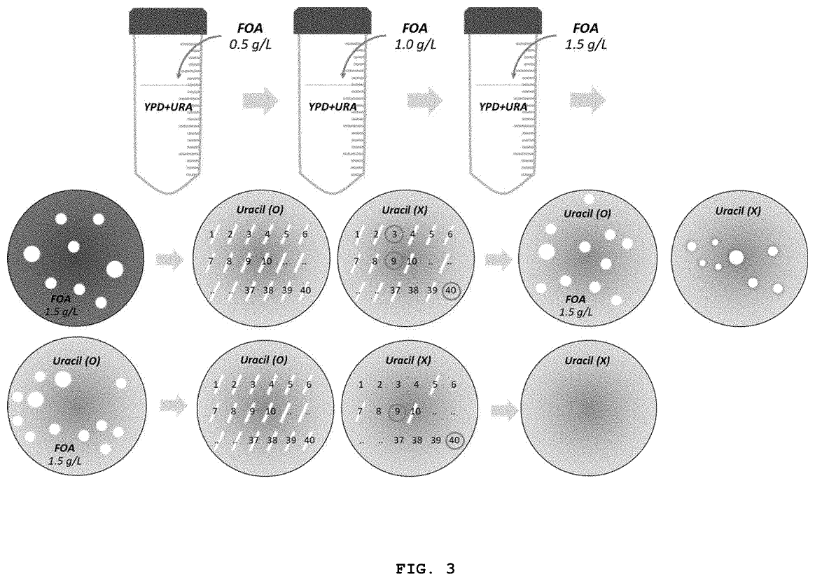

Wickerhamomyces ciferrii ATCC 14091 was used as a parent strain to construct a uracil auxotrophic strain named Y94 obtained by 5-FOA (fluoroorotic acid) random mutagenesis via 5-FOA. To construct the uracil auxotrophic strain Y94, 0.1 g/L 5-FOA was added to a YEPD medium, into which the wild-type (WT) strain was then inoculated. After the strain grew, the concentration of FOA in the medium was gradually increased, and a strain grown in media containing 1.5 g/L FOA was obtained. The obtained strain was streaked on a YEPD agar plate containing 1.5 g/L FOA. Forty colonies were then selected and streaked on the master plate containing SD plates with and without uracil. In addition, colonies that grew on a plate without uracil were selected. These grown colonies were re-streaked on a medium containing 5-FOA in the same manner as above and grown colonies were selected. Through this process, a strain that grew on the SD plate with uracil but did not grow on the plate without uracil were obtained. Finally, it was confirmed that the strain thus obtained did not grow in the liquid medium without uracil (FIG. 3).

Meanwhile, E. coli DH5a was used as a host for gene cloning. To construct pSH471, egfp was introduced into the pSH47 plasmid using the primer set of egfp_for and egfp_rev. Thereafter, the PCR products of egfp and pSH47 were cut using HindIII and EcoRI, and ligated using NEBuilder HiFi DNA Assembly Master Mix (New England Biolabs, MA, USA). Replication origin tests were performed using plasmids pSH471 containing the URA3 selection marker and pSH472 containing the KanMX marker. To construct the pSH472 plasmid, the primer set of KanMX_for and KanMX_rev was used in the pML104 plasmid and ligation was performed using NEBuilder HiFi DNA Assembly Master Mix. Next, the plasmid with the CEN/ARS origin was used as a reference, and the primer set of ARS_for and ARS_rev and the primer set of 2p_for and 2p rev were used to amplify ARS origin and 2p origin sequences, respectively. ARS was amplified from the S. cerevisiae genome sequence, and the 2p origin was amplified from the pML104 plasmid. To construct pEXP, the p416GPD plasmid was used as a backbone. For overexpression of LCB1, LCB2, and SYR2, the ENO1 promoter, TDH3 promoter, and GPD promoter were used, respectively. First, gene fragments were amplified by PCR using the primer set of ENO1_for and ENO1_rev/LCB1_for, the primer set of LCB1_rev/TDH3_for and TDH3_rev/LCB2_for, and the primer set of LCB2_rev/SYR2_for and SYR2_rev (Table 2).

The amplified DNA fragments were then inserted into the p416GPD plasmid, cut by EagI, using NEBuilder HiFi DNA Assembly Master Mix. To construct the LCB4 deletion vector, the pCM184 plasmid was used as a backbone. To perform gene deletion in the Y94 strain, the URA3 selection marker was introduced into pCM184 to construct pCM1841. For the construction of pCM1841, URA3 was amplified using the primer set of EO_for and EO_rev, and pCM184 was cut using PstI. Then, the two fragments were ligated using NEBuilder HiFi DNA Assembly Master Mix. After constructing pCM1841, the left homology region (LHR) of LCB4 was amplified using the primer set of LCB4_LHR_for and LCB4_LHR_rev. The pCM1841 and LHR fragments were cut with EcoRI and KpnI and ligated using Mighty Mix (Takara, Shiga, Japan). Then, the right homology region (RHR) of LCB4 was inserted into pCM1841 containing the LHR of LCB4. The vector was cut with SacI and ligated with the PCR product of RHR, amplified using the primer set of LCB4_RHR_for and LCB4_RHR_rev, by NEBuilder HiFi DNA Assembly Master Mix. These PCR products were amplified using Q5 High-Fidelity DNA Polymerase (New England Biolabs, MA, USA) in a T100 Thermal Cycler (Bio-rad, CA, USA).

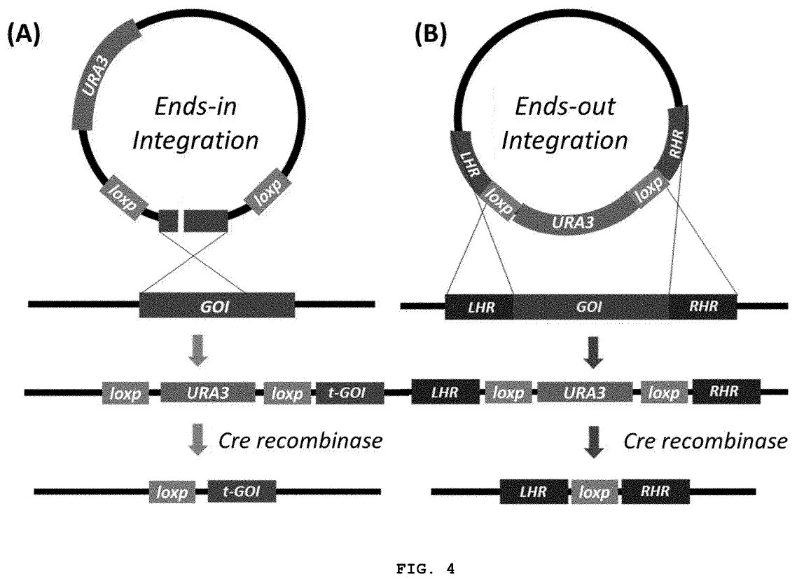

Here, all restriction enzymes were purchased from New England Biolabs. In addition, all vectors were transformed using an Eporator (Eppendorf, Hamburg, Germany) at a voltage of 2,500 V. W. ciferrii YP1 and YP11 were obtained following transformation of pEXP into Y94. After transformation of pEXP, 12 colonies were precultured with the control strains WT and Y94. Then, YP1 and YP11 showing a higher TAPS titer than those of WT and Y94 were selected. The gene LCB4 was deleted using the cre-loxP system via the ends-out method using pCM1842 with homology arms to generate YD94 (FIG. 4). Subsequently, pEXP was transformed into YD94. As described above, 12 colonies were pre-cultured, and YPD7 which showed improved TAPS production was obtained.

### Experimental Example 2. Medium and Culturing

E. coli DH5a for gene cloning was cultured in Luria-Bertani (LB) broth (5 g/L yeast extract, 10 g/L tryptone, and 10 g/L NaCl) at 37° C. 50 μg/L carbenicillin was supplemented for selection. Wickerhamomyces ciferrii wild-type seed strains were grown in 3-ml YEPD medium (10 g/L Bacto yeast extract, 20 g/L Bacto peptone, and 100 ml/L 20% D-glucose). Uracil auxotrophic strains were grown in SD medium (6.7 g/L YNB without amino acids, and 100 ml/L 20% D-glucose) containing a selected amino acid mixture (10 ml/L L-histidine, L-leucine, and L-tryptophan). Then, the seed strains were inoculated into 5 ml of TAPS medium (100 g/L glycerol, 2 g/L yeast extract, 3 g/L KNO₃, 3 g/L sodium acetate, 1 g/L NH4NO3, and 1.5 g/L corn steep powder) and cultured for 1.5 days. After pre-culturing in TAPS medium, the strains were re-inoculated into 50 ml of fresh TAPS medium with OD₆₀₀ of 0.1 for main culture. All culture procedures were performed aerobically at 30° C. in a shaking incubator at 250 rpm.

Fed-batch fermentation of Wickerhamomyces ciferrii YPD7 was performed in 3-L bioreactors in a volume of 1 L TAPS medium containing 100 g/L glycerol, 1 g/L KH₂PO₄, and 1 g/L MgSO₄ for 7 days. The initial OD₆₀₀ for fermentation was 1.0. The fed-batch fermentation was performed at 30° C. with an aeration rate of 1 L/min and an agitation speed of 650 rpm. The aeration rate and agitation speed were increased to 5 L/min and 800 rpm, respectively, to maintain the dissolved oxygen level at 30% or higher. For additional feeding, after confirming the residual concentration of glycerol, 1,000 g/L glycerol feedstock was added to maintain a glycerol concentration of 100 g/L. In addition, 10 mL of 100 g/L amino acids (glutamate and glycine), 10 mL of 200 g/L yeast extract, 10 mL of 100 g/L MgSO₄, and 10 mL of 100 g/L KH₂PO₄) were added every 24 hours.

### Experimental Example 3. Analytical Method

The growth of strains was analyzed by measuring the optical density (OD₆₀₀) using a DU730 UV-Vis spectrophotometer (Beckman Coulter, Brea, CA, USA). EGFP expression was measured using a BioTek Synergy H1 microplate reader (BioTek, VT, US). The glycerol concentration in the medium was measured using high-performance liquid chromatography (HPLC) equipped with a Waters 2414 refractive index detector (Milford, Milford, MA, USA) and Shodex SH1011 column (Shodex, Tokyo, Japan) after centrifuging the culture broth. 10 mM H₂SO4 was used as the mobile phase for HPLC at a flow rate of 0.6 mL/min.

Then, for TAPS analysis, 250 μL of each liquid culture sample was used to measure TAPS production. Specifically, the sample was mixed with 1 mL of acetone and vortexed for 30 min. Next, 700 μL of the supernatant was collected by centrifugation, and then the collected supernatant was analyzed using HPLC equipped with a Waters 2695 HPLC (Waters, Milford, MA), Agilent XDB-C18 column (Agilent, Santa Clara, CA), and Waters 2487 detector (Waters, Milford, MA). The mobile phase was composed of methanol (81.5% w/w), H₂O (18.45%, w/w), and trifluoroacetic acid (0.05% w/w) and applied at a flow rate of 1.4 mL/min for 30 min/run. Here, the UV detection wavelength was 200 nm.

### MODE FOR INVENTION

### Example 1. Establishment of Expression Plasmid for Wickerhamomyces ciferrii

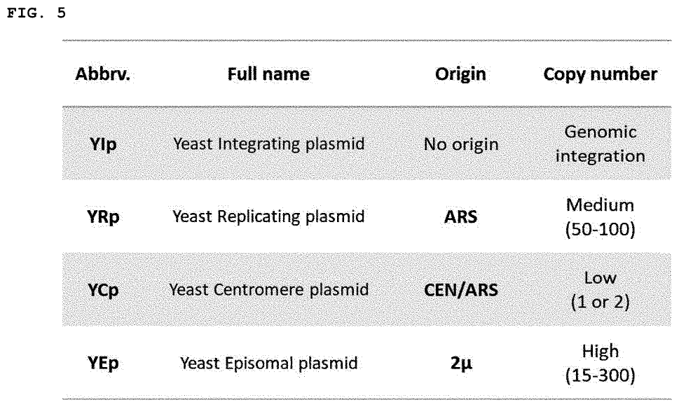

There are four types of plasmids used in the genetic engineering of yeast: yeast replicating plasmids (YRp) with an ARS origin, yeast centromere plasmids (YCp) with a CEN/ARS origin, yeast episomal plasmids (YEp) with a 2p origin, and yeast integration plasmids (YIp) which have no origin and can be integrated directly through homologous recombination in the host chromosome (FIG. 5).

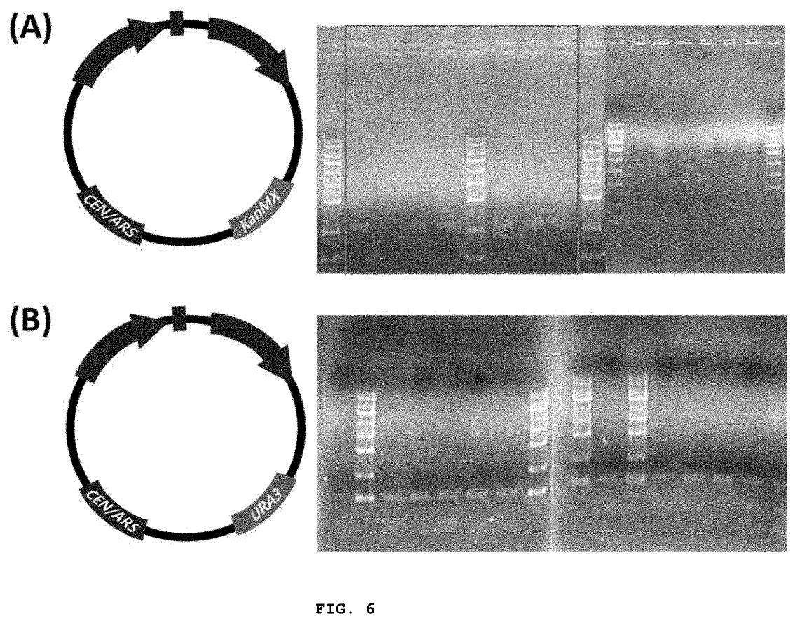

Therefore, the present inventors first examined which origin could function in Wickerhamomyces ciferrii. Specifically, three plasmids with ARS, CEN/ARS, and 2μ origins were each transformed into Wickerhamomyces ciferrii WT. The plasmids were derived from pSH47 containing the KanMX selection marker. Since clony PCR is not applicable to Wickerhamomyces ciferrii, gDNA was extracted to check whether it contained a plasmid. Forward and reverse primers were used as the sequences flanking the replication origin, and PCR was performed to determine whether Wickerhamomyces ciferrii possessed the plasmid. As a result, as can be seen in FIG. 6A, it could be confirmed that the plasmid with the ARS origin and the plasmid with the 2μ origin were not present in Wickerhamomyces ciferrii, but only the plasmid with the CEN/ARS origin was retained (FIG. 6A).

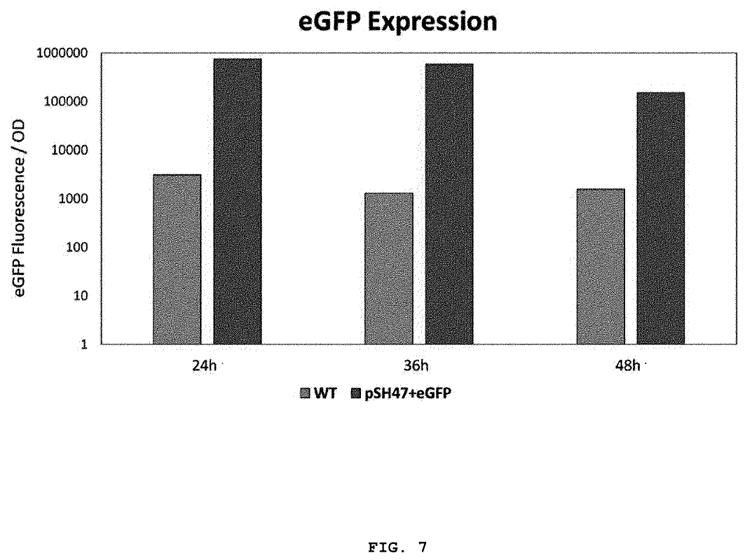

Next, egfp encoding enhanced green fluorescent protein was expressed and the fluorescence level was measured to confirm whether the gene in the plasmid was stably expressed within the strain. As a result, as can be seen in FIG. 7, it was confirmed that egfp was stably expressed within the strain (FIG. 7).

Meanwhile, the KanMX selection marker, which is resistant to kanamycin/G418, is an antibiotic marker used in yeast. However, the selection marker has several disadvantages, such as low selection efficiency and instability. Therefore, there is a need to construct a new and efficient selection marker. Typically, 5-fluoroorotic acid (5-FOA) is used in yeast genetics to confirm the absence of the URA3 gene, because URA3 encodes the enzyme that catalyzes the decarboxylation of 5-FOA to the toxic metabolite 5-fluorouracil, leading to death (Boeke et al., 1987, Luo et al., 2021, Shivhare et al., 2021). Using this 5-FOA, a uracil auxotrophic strain was successfully constructed through the screening process of Experimental Example 1. Next, the replication origin test was conducted again using the newly constructed uracil auxotrophic strain. As a result of cloning three origins into the plasmid containing the URA3 selection marker, as can be seen in FIG. 6B, it was could be confirmed again that only the plasmid derived from CEN/ARS was maintained in the strain, similar to the result shown in FIG. 6A. In addition, unlike the case where kanamycin was the selection marker, it was confirmed that bands appeared in all colonies, indicating a transformation efficiency of 100%. Thereby, it could be confirmed that a strain with an efficient and stable selection marker was effectively constructed (FIG. 6B).

### Example 2. Genetic Engineering for TAPS Production

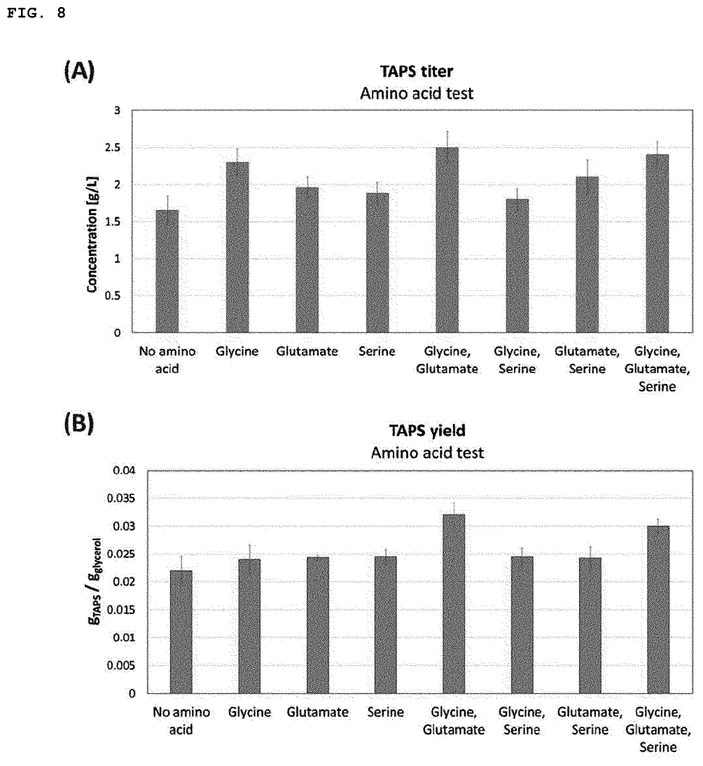

Using the strain constructed in Example 1, an experiment was performed to determine the effect of adding certain amino acids on TAPS production. First, since serine is a precursor of TAPS and glutamate and glycine are most effective in TAPS production, wild-type Wickerhamomyces ciferrii was cultured in TAPS medium to which three amino acids were added (FIG. 8). The amino acids were added to the medium when cells entered the early-mid exponential phase (24 hours). As a result of performing this amino acid test, as can be seen in FIG. 8, it could be confirmed that the TAPS titer was highest in the stationary phase when glutamate and glycine were added together to the medium. In particular, it was confirmed that the TAPS titer of the WT strain was 1.65 g/L in only the TAPS medium, but was 2.5 g/L, which is a 1.5-fold increase, in the TAPS medium containing glutamate and glycine. It could be confirmed that the yield was also highest in the medium containing glutamate and glycine (0.032 g_{TAPS}/g_{glycerol}), but 0.022 g_{TAPS}/g_{glycerol} in WT.

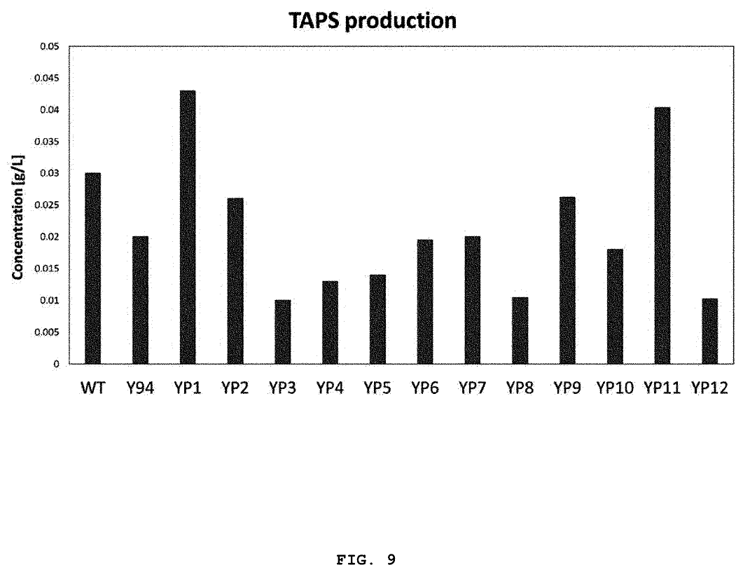

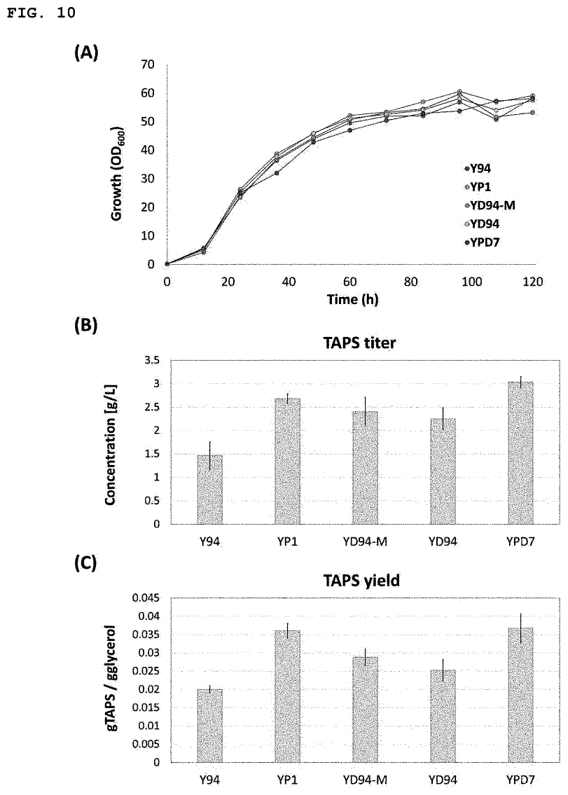

Next, after optimization of medium conditions, Wickerhamomyces ciferrii was engineered using the expression plasmid containing the CEN/ARS origin of replication and the URA3 selection marker to overexpress LCB1, LCB2 and SYR2. Here, LCB1 and LCB2 are genes encoding SPT, and SYR2 is a gene encoding sphinganine C4-hydroxylase. Overexpression of these genes through this promoter engineering in the genome was previously reported to increase TAPS production in S. cerevisiae by 57% (Schorsch et al., 2012). Three strong promoters that can be used in Wickerhamomyces ciferrii were selected to overexpress these genes with p416GPD. LCB1 and LCB2 genes were overexpressed from the ENO1 and TDH3 promoters, respectively, and SYR2 was overexpressed from the GAP promoter. The pEXP expression plasmid with the three genes and three strong different promoters was transformed into the Y94 strain. After transformation, 12 colony candidates were selected for preculture in aerobic tubes. After 4 days of culture, two candidates, YP1 and YP11, which showed better TAPS production than the wild type, were selected (FIG. 9). Culture of YP1 and YP11 was performed in TAPS medium supplemented with glutamate and glycine compared to the control strain (FIG. 10).

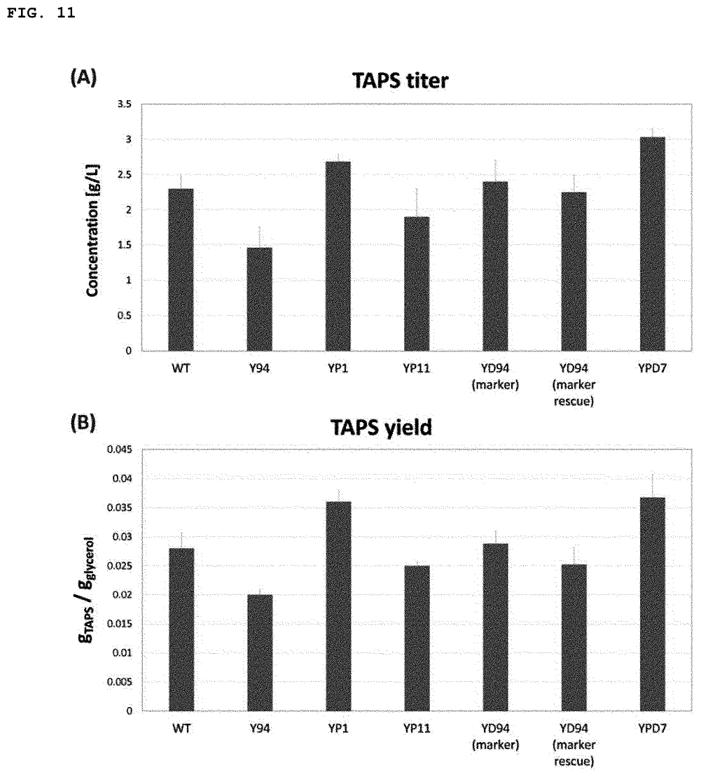

As a result, as can be seen in FIG. 11, the TAPS titers of the WT strain and the Y94 strain were 2.3 g/L and 1.46 g/L, respectively, while the TAPS titers of YP1 and YP11 were 2.68 g/L and 1.9 g/L, respectively. The titer of YP1 was higher than those of the wild type and Y94. It could be confirmed that the yield also increased by 0.036 g_{TAPs}/g_{glycerol}, which corresponds to a 1.8-fold increase compared to Y94 (FIG. 11).

Furthermore, to further increase TAPS production, LCB4 encoding long-chain base kinase was deleted using the cre-loxP system to generate YD94.

There are two methods for replacing target genes based on homologous recombination: the ends-in and ends-out methods. Thereamong, the ends-out method is mainly used, and thus a deletion system was constructed using the ends-out method. To establish the deletion method, the pCM184 plasmid was used with homology arms for the target gene together with loxP sites containing the URA3 selection marker, and the pSH47 plasmid containing Cre recombinase was used with the KanMX selection marker. As a result, it could be confirmed that the TAPS titer of YD94 was 2.25 g/L and the yield was 0.0252 g_{TAPS}/g_{glycerol}, indicating that TAPS production was higher than that of the parent strain Y94 (FIG. 11). YPD7 was constructed by transforming pEXP into YD94. Next, YPD7 was cultured and the TAPS titer and yield thereof were measured (FIG. 11). As a result, it could be confirmed that the TAPS titer and yield of YPD7 were 3.03 g/L and 0.03675 g_{TAPs}/g_{glycerol}, respectively, which were two-fold higher than those of the parent strain Y94. For reference, the YPD7 strain constructed here was deposited with the Korea Research Institute of Bioscience and Biotechnology on May 4, 2022 under accession number KCTC14970BP.

In conclusion, it could be confirmed that overexpression using an expression plasmid harboring a CEN/ARS origin and a URA3 selection marker as well as deletion using the cre-loxP system in Wickerhamomyces ciferrii effectively improved TAPS production.

### Example 3. Fed-Batch Fermentation for TAPS Production

To achieve high TAPS production, high-cell-density fermentation was performed using YPD7 as the final strain. The culture method was fed-batch culture, and glycerol, glutamate, and glycine were periodically added to the culture medium. In addition, nutrients such as MgSO₄, which increases lipid production, and KH₂PO₄, which is a phosphorus (P) source, were supplemented to promote growth. To block foam formation, an antifoaming agent was connected to the fermenter and supplied automatically.

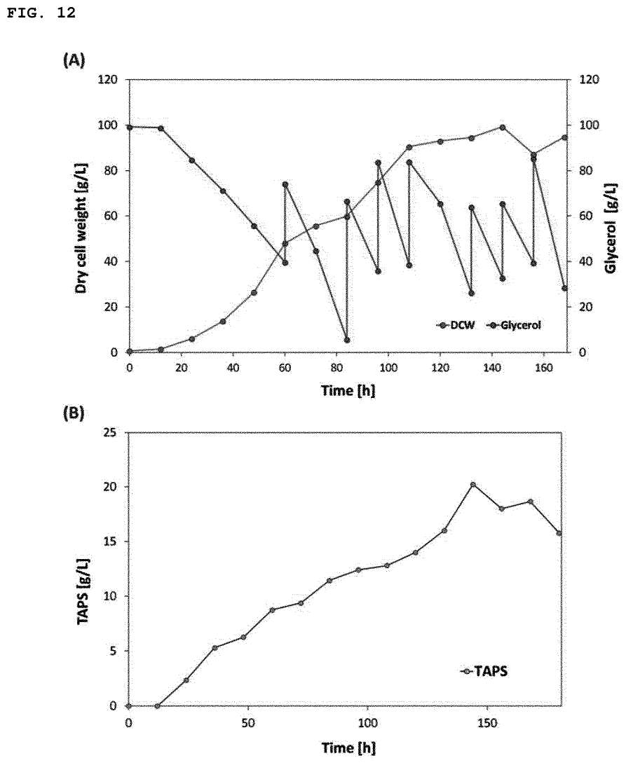

As a result, as can be seen in FIG. 12, it was confirmed that the highest TAPS titer was 20.2 g/L and the productivity was 0.12 g/L/h (FIG. 12).

Thereby, it was confirmed that fed-batch fermentation by the periodic addition of glycerol, glutamate, glycine, MgSO₄, and KH₂PO₄ achieved the highest TAPS titer compared to previous studies (Table 3).

TABLE 3

| | TAPS | | TAPS | | |
|---|---|---|---|---|---|
| Strain | titer | | productivity | | Reference |
| W. ciferrii | 2 | g/L | 0.02 | g/L/h | (Schorsch |
| CSS.L4.O.L2.L1.S2 | | | | | et al., 2012) |
| W. ciferrii | 17.7 | g/L | 0.1475 | g/L/h | (Choi et |
| Mutant 736 | | | | | al., 2021) |
| W. ciferrii | 7.2 | g/L | 0.06 | g/L/h | (Hong & |
| ATCC 14091 | | | | | Yu, 2003) |
| W. ciferrii | 9.65 | g/L | 0.057 | g/L/h | (Hong & |
| UV-P63-NTG4 | | | | | Yu, 2002) |
| W. ciferrii | 5.66 | g/L | 0.03 | g/L/h | (Dae-iL OH, |
| DSCC7-25 | | | | | 2004) |
| W. ciferrii | 20.2 | g/L | 0.12 | g/L/h | This study |
| YD7 strain | | | | | |

In summary, in order to express a gene in a simple and effective manner by constructing an effective expression plasmid that can work in Wickerhamomyces ciferrii, various replication origin tests previously used in yeast were performed to find a CEN/ARS origin, and in order to obtain an efficient selection marker, a uracil auxotrophic Wickerhamomyces ciferrii strain was constructed using 5-FOA. Next, the gene to be overexpressed was cloned into an expression plasmid containing the CEN/ARS origin and the uracil selection marker, and then the expression plasmid was transformed into the constructed uracil auxotrophic Wickerhamomyces ciferrii strain, and the strain YP1 with the highest TAPS production was selected. It could be seen that the final titer of YP1 was 2.68 g/L, which was 1.8 times higher than the titer of the uracil auxotrophic strain, 1.46 g/L. In addition, it was confirmed that the TAPS titer of YPD7 obtained by deleting the LCB4 gene was 3.03 g/L, which was 2 times higher than that of the control group. Finally, as a result of optimizing the medium conditions by adding amino acids, which are precursors involved in serine synthesis, to the medium, it was confirmed that the TAPS titer in the final strain YPD7 was 20 g/L, indicating that TAPS could be produced at a much higher concentration than previously known.

This suggests that, when a uracil auxotrophic Wickerhamomyces ciferrii mutant strain constructed using an expression plasmid having a specific origin of replication and a uracil selection marker is engineered, the TAPS production in fed-batch culture of the strain may be more effectively increased.

While the present invention has been described, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive. The scope of the present invention is defined by the appended claims rather than the detailed description, and it should be understood that all modifications or variations conceived from the meanings and scope of the appended claims and equivalents thereto are included in the scope of the present invention.

### [Accession Number]

Depository authority: Korea Research Institute of Bioscience and Biotechnology
Accession number: KCTC14970BP
Deposit date: May 4, 2022

### INDUSTRIAL APPLICABILITY

The Wickerhamomyces ciferrii mutant strain according to the present invention has excellent TAPS productivity, and thus may be advantageously used in various fields in which TAPS is used.

1. An expression plasmid for genetic engineering of Wickerhamomyces ciferrii, containing a CEN/ARS origin of replication and a uracil selection marker.

2. The expression plasmid of claim 1, wherein the uracil selection marker is 5-FOA.

3. The expression plasmid of claim 1, further containing a gene encoding serine palmitoyl-transferase and a gene encoding sphinganine C4-hydroxylase.

4. The expression plasmid of claim 3, wherein the gene encoding serine palmitoyl-transferase is LCB1, LCB2, or a combination thereof.

5. The expression plasmid of claim 4, wherein the gene encoding sphinganine C4-hydroxylase is SYR2.

6. The expression plasmid of claim 3, wherein the expression plasmid is pEXP.

7. The expression plasmid of claim 1, further containing homologous arms of a gene encoding a long-chain base kinase.

8. The expression plasmid of claim 7, wherein the gene encoding the long-chain base kinase is LCB4.

9. The expression plasmid of claim 8, wherein the expression plasmid is pCM1842.

10. A Wickerhamomyces ciferrii strain having an improved ability to produce tetraacetylphytosphingosine (TAPS), obtained by enhancing activities of serine palmitoyl-transferase and sphinganine C4-hydroxylase by transformation with the expression plasmid of any one of claims 3 to 6, and weakening an activity of a long-chain base kinase using the expression plasmid of any one of claims 7 to 9.

11. The Wickerhamomyces ciferrii strain of claim 10, wherein the Wickerhamomyces ciferrii strain is deposited under accession number KCTC14970BP.

12. A Wickerhamomyces ciferrii strain having an improved ability to produce tetraacetylphytosphingosine (TAPS), obtained by enhancing activities of serine palmitoyl-transferase and sphinganine C4-hydroxylase in a uracil auxotrophic Wickerhamomyces ciferrii strain containing a uracil selectable marker using an expression plasmid containing a CEN/ARS origin of replication and further weakening an activity of a long-chain base kinase.

13. The Wickerhamomyces ciferrii strain of claim 12, wherein the serine palmitoyl-transferase is encoded by LCB1 or LCB2.

14. The Wickerhamomyces ciferrii strain of claim 12, wherein the sphinganine C4-hydroxylase is encoded by SYR2.

15. The Wickerhamomyces ciferrii strain of claim 12, wherein the long-chain base kinase is encoded by LCB4.

16. The Wickerhamomyces ciferrii strain of claim 12, wherein the Wickerhamomyces ciferrii strain is deposited under accession number KCTC14970BP.

17. A composition for producing tetraacetylphytosphingosine (TAPS), comprising the strain of any one of claims 12 to 16, a culture of the strain, a lysate of the strain, or an extract of the strain.

18. A method for producing tetraacetylphytosphingosine (TAPS), comprising a step of culturing the strain of any one of claims 12 to 16 in a medium.

19. A method for constructing a Wickerhamomyces ciferrii strain having an improved ability to produce tetraacetylphytosphingosine (TAPS), comprising:
a first step of constructing a uracil auxotrophic Wickerhamomyces ciferrii strain using 5-FOA;
a second step of enhancing activities of serine palmitoyl-transferase and sphinganine C4-hydrosylase using an expression plasmid containing a CEN/ARS origin of replication; and
a third step of weakening an activity of a long-chain base kinase.
